(19) United States
(12) Patent Application Publication
(10) Pub. No.: US 2023/0102448 A1
(43) Pub. Date: Mar. 30, 2023

(54) PHOTOACTIVE MATERIAL

(71) Applicant: Sumitomo Chemical Co., Ltd., Tokyo (JP)

(72) Inventors: Nir Yaacobi-Gross, Godmanchester (GB); Ben Gardner, Godmanchester (GB)

(73) Assignee: Sumitomo Chemical Co., Ltd., Tokyo (JP)

(21) Appl. No.: 17/797,235

(22) PCT Filed: Jan. 29, 2021
(86) PCT No.: PCT/GB2021/050204

§ 371 Date: Aug. 3, 2022

(30) Foreign Application Priority Data
Feb. 4, 2020 (GB) 2001499.9
Jul. 29, 2020 (GB) 2011802.2

(51) Int. Cl. H01L 51/00; H01L 51/42

(52) U.S. Cl. CPC H01L 51/0036; H01L 51/0043; H01L 51/4253

(57) ABSTRACT

A material comprising an electron-accepting unit of formula (I): wherein Ar1 and Ar2 independently is a 5- or 6-membered aromatic or heteroaromatic ring or is absent; and each X is independently H or a substituent with the proviso that at least one X is an electron-withdrawing group and wherein X groups bound to adjacent carbon atoms may be linked to form an electron-withdrawing group. The material further comprises an electron-donating unit D comprising a fused or unfused furan or thiophene. The material may be a polymer comprising repeat units of formula (I). The material may be a non-polymeric compound. An organic photodetector may contain a bulk heterojunction layer containing an electron acceptor or an electron donor wherein at least one of the electron acceptor and electron donor contains a unit of formula (I).

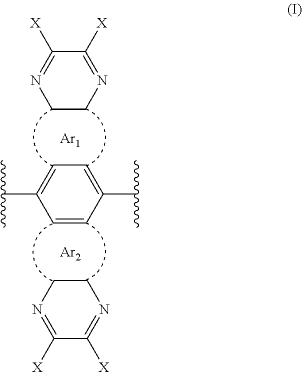

### BACKGROUND

Embodiments of the present disclosure relate to photoactive materials and more specifically, but not by way of limitation, to photoactive materials containing an electron-accepting unit and an electron-donating unit, the materials being suitable for us as an electron-donating material or an electron-accepting material in a photoresponsive device.

Wu, WC. & Chen, WC. “Theoretical Electronic Structure and Properties of Alternating Fluorene-Acceptor Conjugated Copolymers and Their Model Compounds”, J Polym Res (2006) 13: 441, discloses the theoretical geometries and electronic properties of fluorene (F) based alternating donor-acceptor conjugated copolymers with a range of acceptors including pyrazinoquinoxaline.

Kai-Fang Cheng et al, “New fluorene-pyrazino[2,3-g]quinoxaline-conjugated copolymers: Synthesis, optoelectronic properties, and electroluminescence characteristics”, J. Appl. Poly. Sci., Vol. 112, Issue 4, 15 May 2009, p. 2094-2101 discloses donor-acceptor conjugated copolymers of poly2,7-(9,9′-dihexylfluorene)-co-5,10-[pyrazino(2,3-g)quinoxaline].

Unver et al, “Synthesis of new donor-acceptor polymers containing thiadiazoloquinoxaline and pyrazinoquinoxaline moieties: low-band gap, high optical contrast, and almost black colored materials”, Tetrahedron Letters, Volume 52, Issue 21, 25 May 2011, Pages 2725-272 discloses poly[4,9-bis(4-hexylthien-2-yl)-6,7-di(thien-2-yl)-[1,2,5]thiadiazolo[3,4-g]quinoxaline] (PHTTQ) and poly[5,10-bis(4-hexylthien-2-yl)-2,3,7,8-tetra(thien-2-yl)pyrazino[2,3-g]quinoxaline] (PHTPQ), consisting of alternating electron-rich 3-hexylthiophene and electron-deficient 6,7-di(thien-2-yl)-[1,2,5]thiadiazolo[3,4-g]quinoxaline (TTQ) and 2,3,7,8-tetra(thien-2-yl)-2,3-dihydropyrazino[2,3-g]quinoxaline (TPQ) units.

KR 20180042966 discloses an OLED containing an organic light-emitting compound of formula 1:

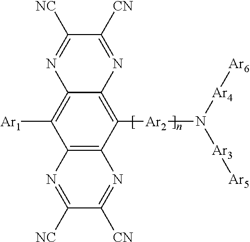

According to some embodiments, the present disclosure provides a material comprising an electron-accepting unit of formula (I):

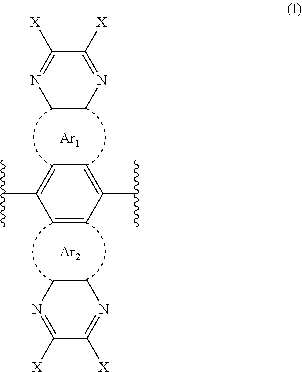

wherein Ar¹ is a 5- or 6-membered aromatic or heteroaromatic ring or is absent; Ar² is a 5- or 6-membered aromatic or heteroaromatic ring or is absent; and each X is independently H or a substituent with the proviso that at least one X is an electron-withdrawing group and wherein X groups bound to adjacent carbon atoms may be linked to form an electron-withdrawing group; the material further comprising an electron-donating unit D comprising a fused or unfused furan or thiophene.

Optionally, each X is an electron-withdrawing group.

Optionally, the or each electron-withdrawing group is selected from:
a group R⁴ wherein each R⁴ is independently selected from the Cl, CN, NO₂, COOR³, C₁₋₆ fluoroalkyl, e.g. —CF₃, —OR³, —SR³, —SO₂R³, —SO₃R³, —CHO, —C(O)R³, —C(S)R³, —C(S)OR³, —OC(O)R³, —OC(S)R³, —C(O)SR³, —SC(O)R³, —C(O)NR³₂, —NRC(O)R³, —CH═CH(CN), —CH═C(CN)₂, —C(CN)═C(CN)₂, —CH═C(CN)(R³), —CH═C(CN)C(O)OR³ and —CH═C(CONR³₂)₂, wherein R³ is H or a substituent; and
phenyl substituted with one or more R⁴ groups.

Optionally, the material is a non-polymeric compound. Optionally, non-polymeric compound has formula (Ia) or (Ib):

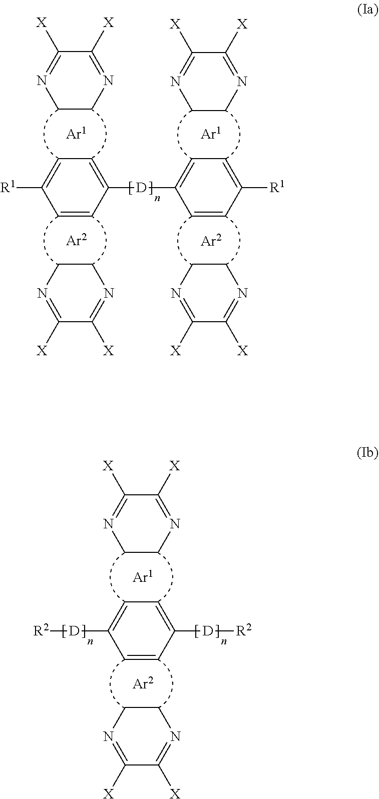

wherein n is at least 1; and R¹ and R² independently in each occurrence is H or a substituent.

Optionally, the material is a polymer; the unit of formula (I) is an electron-accepting repeat unit of formula (I); and the electron-donating unit D is an electron-donating repeat unit.

Optionally, D of a non-polymeric compound or a repeat unit D of a polymer as described herein is selected from formulae (IIa)-(IIo):

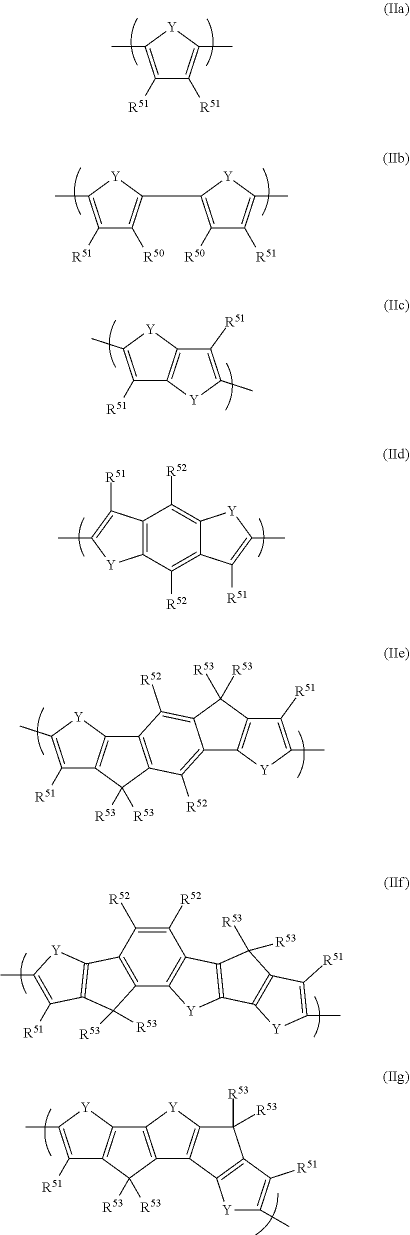

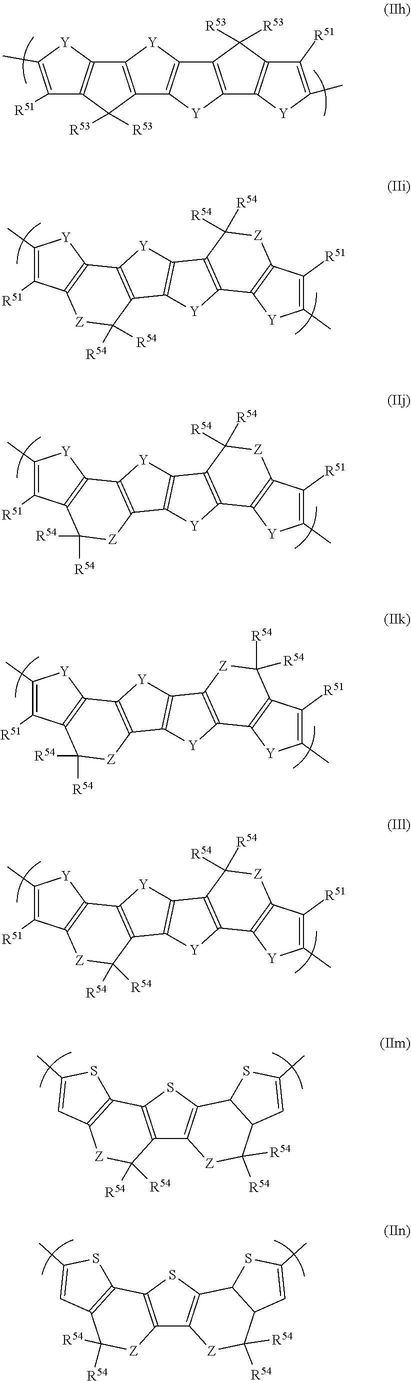

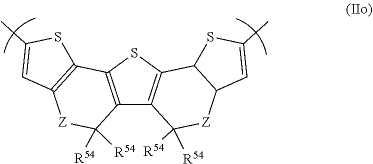

wherein Y in each occurrence is independently O or S, Z in each occurrence is O, S, NR⁵⁵ or C(R⁵⁴)₂; R⁵⁰, R⁵¹, R⁵² and R⁵⁴ and R⁵⁵ independently in each occurrence is H or a substituent wherein R⁵⁰ groups may be linked to form a ring; and R⁵³ independently in each occurrence is a substituent.

According to some embodiments, the present disclosure provides a polymer comprising a repeat unit of formula (I):

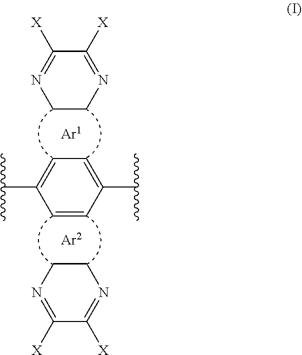

wherein Ar¹ is a 5- or 6-membered aromatic or heteroaromatic ring or is absent; Ar² is a 5- or 6-membered aromatic or heteroaromatic ring or is absent; and each X is independently H or a substituent with the proviso that at least one X is an electron-withdrawing group and wherein X groups bound to adjacent carbon atoms may be linked to form an electron-withdrawing group. The polymer may contain donor repeat units D as described anywhere herein.

According to some embodiments, the present disclosure provides a composition comprising an electron donor and an electron acceptor wherein at least one of the electron donor and electron acceptor is a material or polymer as described herein.

In some embodiments, the electron acceptor of the composition is the material comprising an electron-accepting unit of formula (I) as described herein. Optionally, the electron acceptor is a non-polymeric compound as described herein.

In some embodiments, the electron donor is the material comprising an electron-accepting unit of formula (I) as described herein, or a polymer comprising a repeat unit of formula (I) as described herein. Optionally, the electron donor is a polymer as described herein.

According to some embodiments, the present disclosure provides an organic electronic device comprising an active layer comprising a material or composition as described herein.

Optionally, the organic electronic device is an organic photoresponsive device comprising a bulk heterojunction layer disposed between an anode and a cathode and wherein the bulk heterojunction layer comprises a composition as described herein.

Optionally, the organic photoresponsive device is an organic photodetector.

According to some embodiments, the present disclosure provides a photosensor comprising a light source and an organic photodetector as described herein, wherein the photosensor is configured to detect light emitted from a light source. Optionally, the light source emits light having a peak wavelength of at least 900 nm.

According to some embodiments, the present disclosure provides a formulation comprising a material, polymer or composition as described herein dissolved or dispersed in one or more solvents.

According to some embodiments, the present disclosure provides a method of forming an organic electronic device as described herein, wherein formation of the active layer comprises deposition of a formulation as described herein onto a surface and evaporation of the one or more solvents.

### DESCRIPTION OF DRAWINGS

The disclosed technology and accompanying FIGURES describe some implementations of the disclosed technology.

FIG. 1 illustrates an organic photoresponsive device according to some embodiments.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers are may be present. References to a layer “on” another layer when used in this application means that the layers are in direct contact. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

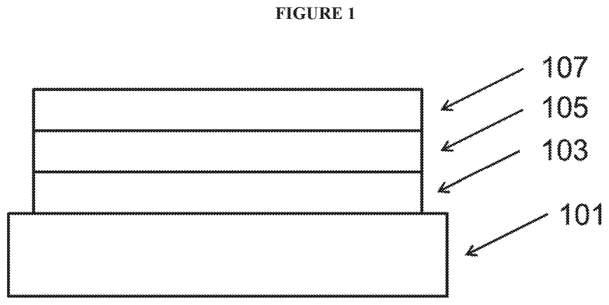

FIG. 1 illustrates an organic photoresponsive device according to some embodiments of the present disclosure. The organic photoresponsive device comprises a cathode 103, an anode 107 and a bulk heterojunction layer 105 disposed between the anode and the cathode. The organic photoresponsive device may be supported on a substrate 101, optionally a glass or plastic substrate.

Each of the anode and cathode may independently be a single conductive layer or may comprise a plurality of layers.

The organic photoresponsive device may comprise layers other than the anode, cathode and bulk heterojunction layer shown in FIG. 1. In some embodiments, a hole-transporting layer is disposed between the anode and the bulk heterojunction layer. In some embodiments, an electron-transporting layer is disposed between the cathode and the bulk heterojunction layer. In some embodiments, a work function modification layer is disposed between the bulk heterojunction layer and the anode, and/or between the bulk heterojunction layer and the cathode.

The area of the OPD may be less than about 3 cm², less than about 2 cm², less than about 1 cm², less than about 0.75 cm², less than about 0.5 cm² or less than about 0.25 cm². The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

The bulk heterojunction layer comprises an electron donor material and an electron acceptor material wherein at least one of the electron donor material and the electron acceptor material comprises an electron-accepting group of Formula (I):

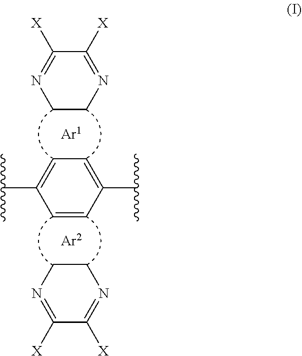

wherein Ar¹ is a 5- or 6-membered aromatic or heteroaromatic ring or is absent; Ar² is a 5- or 6-membered aromatic or heteroaromatic ring or is absent; and each X is independently H or a substituent with the proviso that at least one X is an electron-withdrawing group; the material further comprising an electron-donating unit D comprising a fused or unfused thiophene or furan group.

Preferably, each unit of formula (I) is bound directly to at least one electron-donating unit D.

In some embodiments, the material comprising the unit of formula (I) has an absorption peak in the range of 900-1000 nm.

In some embodiments, the material comprising the unit of formula (I) has an absorption peak at above 1000 nm, optionally in the range of 1300-1400 nm.

The electron donor (p-type) material has a HOMO deeper (further from vacuum) than a LUMO of the electron acceptor (n-type) material. Optionally, the gap between the HOMO level of the p-type donor material and the LUMO level of the n-type acceptor material is less than 1.4 eV. The electron donor and electron acceptor may have a type II interface. Unless stated otherwise, HOMO and LUMO levels of materials as described herein are as measured by square wave voltammetry (SWV).

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

The sample is dissolved in Toluene (3 mg/ml) and spun at 3000 rpm directly on to the glassy carbon working electrode.

LUMO=4.8-E ferrocene(peak to peak average)−E reduction of sample(peak maximum).

HOMO=4.8-E ferrocene(peak to peak average)+E oxidation of sample(peak maximum).

A typical SWV experiment runs at 15 Hz frequency; 25 mV amplitude and 0.004 V increment steps. Results are calculated from 3 freshly spun film samples for both the HOMO and LUMO data.

In some embodiments, the bulk heterojunction layer contains only one electron donor material and only one electron acceptor material, at least one of the donor and acceptor comprising an electron-accepting unit of formula (I).

In some embodiments, the bulk heterojunction layer contains two or more electron donor materials and/or two or more electron acceptor materials.

In some embodiments, the weight of the donor material(s) to the acceptor material(s) is from about 1:0.5 to about 1:2. In some preferred embodiments, the weight of the donor material(s) to the acceptor material(s) is from about 1:1.1 to about 1:2. In some preferred embodiments, the weight of the donor material(s) is greater than the weight of the acceptor material(s).

In some embodiments, the material comprising the group of formula (I) is a non-polymeric compound containing at least one unit of formula (I), optionally 1, 2 or 3 units of formula (I) and at least on electron-donating unit D. Preferably, the non-polymeric compound has a molecular weight of less than 5,000 Daltons, optionally less than 3,000 Daltons. Preferably, the non-polymeric compound contains no more than 3 groups of formula (I).

In some embodiments, the material comprising the group of formula (I) is a polymer comprising a repeat unit of formula (I) and electron-donating repeat units, more preferably alternating electron-accepting repeat units of formula (I) and electron-donating repeat units.

Preferably, the polystyrene-equivalent number-average molecular weight (Mn) measured by gel permeation chromatography of the polymer is in the range of about 5×10³ to 1×10⁸, and preferably 1×10⁴ to 5×10⁶. The polystyrene-equivalent weight-average molecular weight (Mw) of the polymer may be 1×10³ to 1×10⁸, and preferably 1×10⁴ to 1×10⁷.

A non-polymeric compound comprising a unit of formula (I) may have formula (Ia) or (Ib):

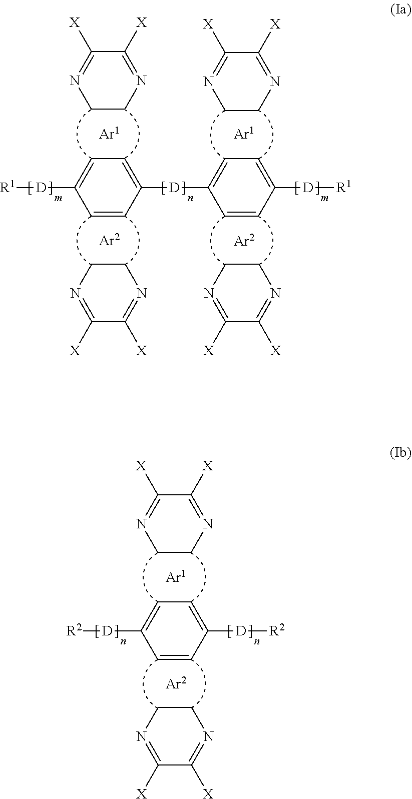

wherein n is at least 1, optionally 1, 2 or 3; m is 0, 1, 2 or 3; D in each occurrence is independently an electron-donating unit comprising a fused or unfused thiophene or furan which may be unsubstituted or substituted with one or more substituents; and R¹ and R² independently in each occurrence is H or a substituent.

Optionally, R¹ and R² are each independently selected from the group consisting of H; F; C₁₋₂₀ alkyl wherein one or more non-adjacent, non-terminal C atoms may be replaced with O, S, COO or CO and one or more H atoms of the alkyl may be replaced with F; and phenyl which is unsubstituted or substituted with one or more substituents, optionally one or more C₁₋₁₂ alkyl groups wherein one or more non-adjacent, non-terminal C atoms may be replaced with O, S, COO or CO and one or more H atoms of the alkyl may be replaced with F.

Ar¹ and Ar², where present, are preferably and independently selected from benzene, thiophene and furan. Ar¹ and Ar² may each independently be unsubstituted or substituted with one or more substituents. Substituents may be selected from non-H groups of R¹ and R² as described above.

A polymer comprising repeat units of formula (I) may contain the a repeating structure of formula (II), comprising the repeat unit of formula (I) and an adjacent electron donating repeat unit D:

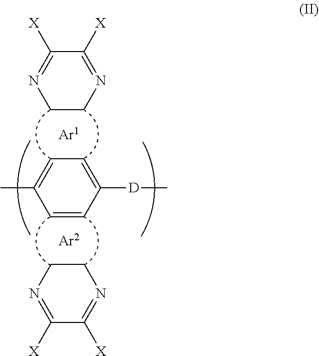

For an electron donor material or electron acceptor material containing an electron accepting unit of formula (I) and an electron-donating unit (D) the, or each, unit of formula (I) has a LUMO level that is deeper (i.e. further from vacuum) than the, or each, electron-donating unit, preferably at least 1 eV deeper. The LUMO levels of an electron-donating unit and an electron-accepting unit of formula (I) may be as determined by modelling, respectively, the LUMO level of D-H or H-D-H and H-[Formula (I)]-H, respectively, i.e. by replacing the bond or bonds between D and Formula (I) with a bond or bonds to a hydrogen atom.

Modelling may be performed using Gaussian09 software available from Gaussian using Gaussian09 with B3LYP (functional).

Preferably, a model compound of formula H-[Formula (I)]-H containing one or more electron-withdrawing groups X has a smaller HOMO-LUMO band gap than a comparative model compound in which each X is H.

Optionally, each electron-withdrawing group X is independently selected from the group consisting of:
a group R⁴ wherein each R⁴ is independently selected from the Cl, CN, NO₂, COOR³, C₁₋₆ fluoroalkyl, e.g. —CF₃, —OR³, —SR³, —SO₂R³, —SO₃R³, —CHO, —C(O)R³, —C(S)R³, —C(S)OR³, —OC(O)R³, —OC(S)R³, —C(O)SR³, —SC(O)R³, —C(O)NR³², —NRC(O)R³, —CH═CH(CN), —CH═C(CN)₂, —C(CN)═C(CN)₂, —CH═C(CN)(R³), —CH═C(CN)C(O)OR³ and —CH═C(CONR³²)₂, wherein R³ is H or a substituent; and
phenyl substituted with one or more R⁴ groups.

Optionally, each R³ is H or a C₁₋₁₂hydrocarbyl group, optionally a C₁₋₁₂ alkyl or phenyl which is unsubstituted or substituted with one or more C₁₋₆ alkyl groups, wherein one or more H atoms of the hydrocarbyl group may be replaced with F.

X groups bound to adjacent carbon atoms may be linked to form an electron-withdrawing ring structure.

Two X groups may be linked to form, without limitation:

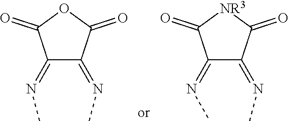

Preferably, each X is independently CN or NO₂.

Electron Donating Unit

Electron-donating units D are preferably in each occurrence a monocyclic or polycyclic heteroaromatic group which contains at least one furan or thiophene and which may be unsubstituted or substituted with one or more substituents. Preferred electron-donating units D are monocyclic thiophene or furan or a polycyclic donor wherein each ring of the polycyclic donor includes thiophene or furan rings and, optionally, one or more of benzene, cyclopentane, or a six-membered ring containing 5 C atoms and one of N and O atoms.

Optionally, electron donating units D are selected from formulae (IIa)-(IIo), or a combination thereof:

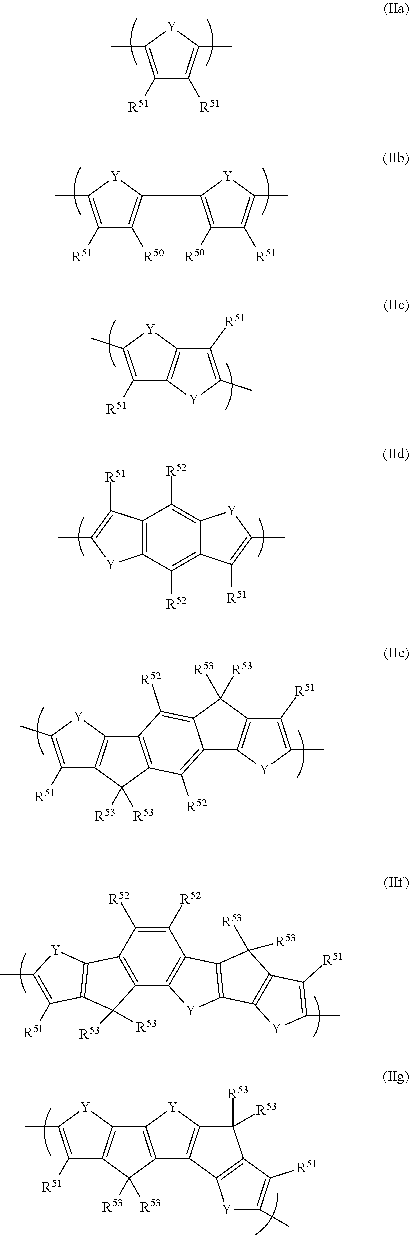

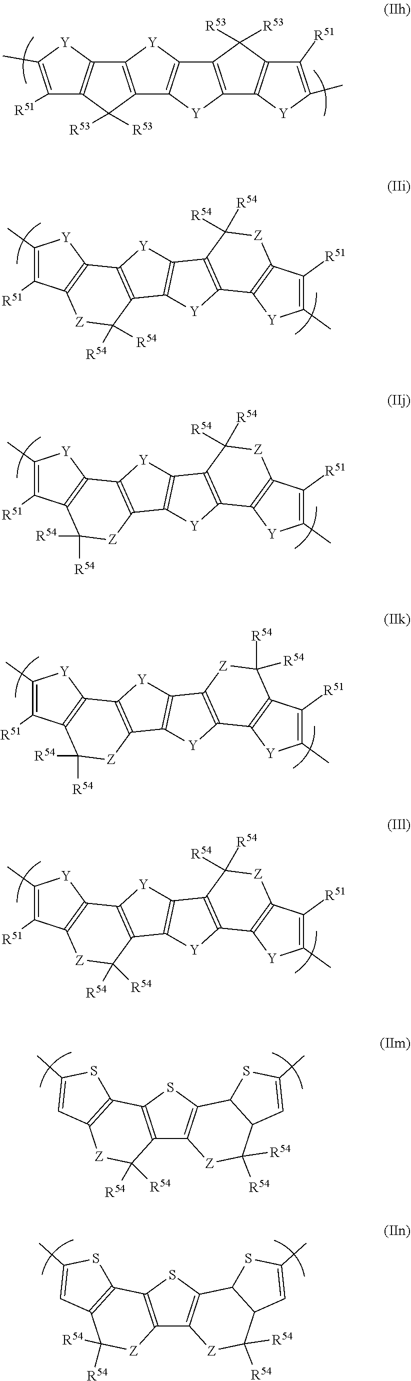

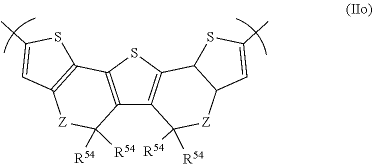

wherein Y in each occurrence is independently O or S, preferably S; Z in each occurrence is O, S, NR⁵⁵, or C(R⁵⁴)₂; R⁵⁰, R⁵¹, R⁵², R⁵⁴ and R⁵⁵ independently in each occurrence is H or a substituent wherein R⁵⁰ groups may be linked to form a ring; and R⁵³ independently in each occurrence is a substituent.

In some embodiments, the electron-donating unit D is a single group of formula (IIa)-(IIo).

In some embodiments, the electron-donating unit D comprises a plurality of directly linked groups of formula (IIa)-(IIo). The directly linked groups may be the same or different.

Optionally, R⁵⁰, R⁵¹ and R⁵² independently in each occurrence are selected from H; F; C₁₋₂₀ alkyl wherein one or more non-adjacent, non-terminal C atoms may be replaced with O, S, COO or CO and one or more H atoms of the alkyl may be replaced with F; and an aromatic or heteroaromatic group Ar³ which is unsubstituted or substituted with one or more substituents.

In some embodiments, Ar³ maybe an aromatic group, e.g. phenyl.

The one or more substituents of Ar³, if present, may be selected from C₁₋₁₂ alkyl wherein one or more non-adjacent, non-terminal C atoms may be replaced with O, S, COO or CO and one or more H atoms of the alkyl may be replaced with F.

By “non-terminal” C atom of an alkyl group as used herein is meant a C atom of the alkyl other than the methyl C atom of a linear (n-alkyl) chain or the methyl C atoms of a branched alkyl chain.

Preferably, each R⁵⁴ is selected from the group consisting of:

H;

linear, branched or cyclic C₁₋₂₀ alkyl wherein one or more non-adjacent, non-terminal C atoms may be replaced by O, S, NR⁷, CO or COO wherein R⁷ is a C₁₋₁₂ hydrocarbyl and one or more H atoms of the C₁₋₂₀ alkyl may be replaced with F; and

a group of formula (Ak)u-(Ar⁴)v wherein Ak is a C₁₋₁₂ alkylene chain in which one or more C atoms may be replaced with O, S, CO or COO; u is 0 or 1; Ar⁴ in each occurrence is independently an aromatic or heteroaromatic group which is unsubstituted or substituted with one or more substituents; and v is at least 1, optionally 1, 2 or 3.

Preferably, each R⁵¹ is H.

Optionally, R⁵³ independently in each occurrence is selected from C₁₋₂₀ alkyl wherein one or more non-adjacent, non-terminal C atoms may be replaced with O, S, COO or CO and one or more H atoms of the alkyl may be replaced with F; and phenyl which is unsubstituted or substituted with one or more substituents, optionally one or more C₁₋₁₂ alkyl groups wherein one or more non-adjacent, non-terminal C atoms may be replaced with O, S, COO or CO and one or more H atoms of the alkyl may be replaced with F.

Preferably, R⁵⁵ is H or a C₁₋₃₀ hydrocarbyl group

Preferably, each R⁵⁰ is a substituent. In a preferred embodiment, the R⁵⁰ groups are linked to form a group of formula —Z—C(R⁵⁴)₂— wherein Z is O, S NR⁵⁵, or C(R⁵⁴)₂, e.g. a group of formula (IIb-1) or (IIb-2):

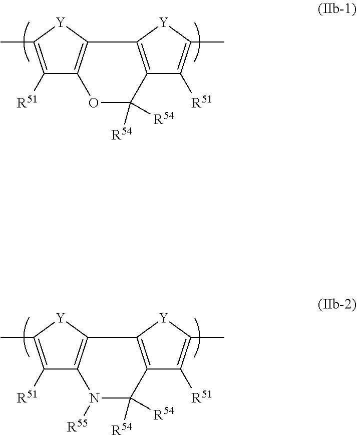

Electron Donor Material

In the case where the material comprising the group of formula (I) is an electron-accepting material, it may be used with any electron donor material containing a group of formula (I) or any other electron donor material known to the person skilled in the art, including organic polymers and non-polymeric organic molecules.

In a preferred embodiment the electron donor material is an organic conjugated polymer, which can be a homopolymer or copolymer including alternating, random or block copolymers. Preferred are non-crystalline or semi-crystalline conjugated organic polymers. Further preferably the p-type organic semiconductor is a conjugated organic polymer with a low bandgap, typically between 2.5 eV and 1.5 eV, preferably between 2.3 eV and 1.8 eV.

Optionally, the p-type donor has a HOMO level no more than 5.5 eV from vacuum level. Optionally, the p-type donor has a HOMO level at least 4.1 eV from vacuum level.

As exemplary p-type donor polymers, polymers selected from conjugated hydrocarbon or heterocyclic polymers including polyacene, polyaniline, polyazulene, polybenzofuran, polyfluorene, polyfuran, polyindenofluorene, polyindole, polyphenylene, polypyrazoline, polypyrene, polypyridazine, polypyridine, polytriarylamine, poly(phenylene vinylene), poly(3-substituted thiophene), poly(3,4-bisubstituted thiophene), polyselenophene, poly(3-substituted selenophene), poly(3,4- bisubstituted selenophene), poly(bisthiophene), poly(terthiophene), poly(bisselenophene), poly(terselenophene), polythieno[2,3-b]thiophene, polythieno[3,2-b]thiophene, polybenzothiophene, polybenzo[1,2-b:4,5-b′jdithiophene, polyisothianaphthene, poly(monosubstituted pyrrole), poly(3,4-bisubstituted pyrrole), poly-1,3,4-oxadiazoles, polyisothianaphthene, derivatives and co-polymers thereof may be mentioned. Preferred examples of p-type donors are copolymers of polyfluorenes and polythiophenes, each of which may be substituted, and polymers comprising benzothiadiazole-based and thiophene-based repeating units, each of which may be substituted. It is understood that the p-type donor may also consist of a mixture of a plurality of electron donating materials.

Optionally, the electron donor polymer comprises a repeat unit selected from formulae (IIa)-(IIf) as described above.

In a preferred embodiment, the repeat units of the electron donor polymer comprise or consist of a repeat unit of formula (I) and a repeat unit of formula (IIb-1) or (IIb-2) in an alternating arrangement as shown in formula (II).

Exemplary electron-donor polymers comprising a repeat unit of formula (I) include polymers having a repeating structure selected from:

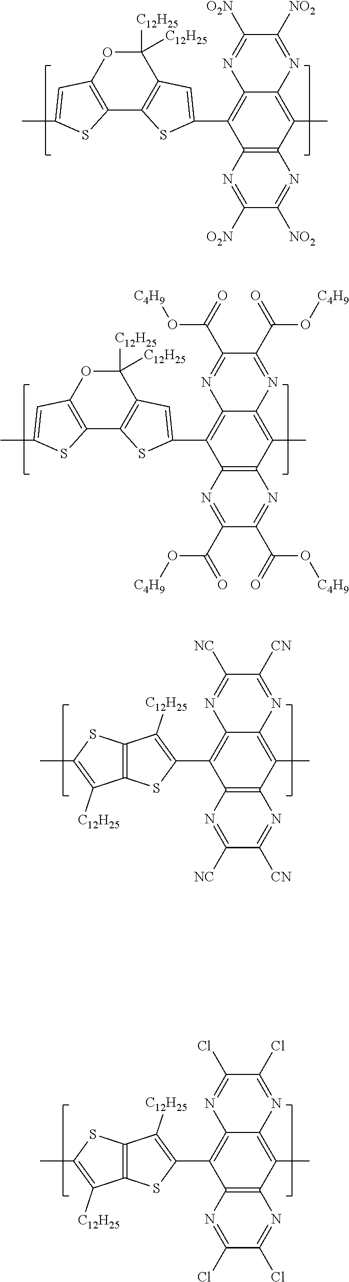

Optionally, in the case where the electron donor polymer does not contain a repeat unit of formula (I), it comprises a repeat unit selected from repeat units of formulae:

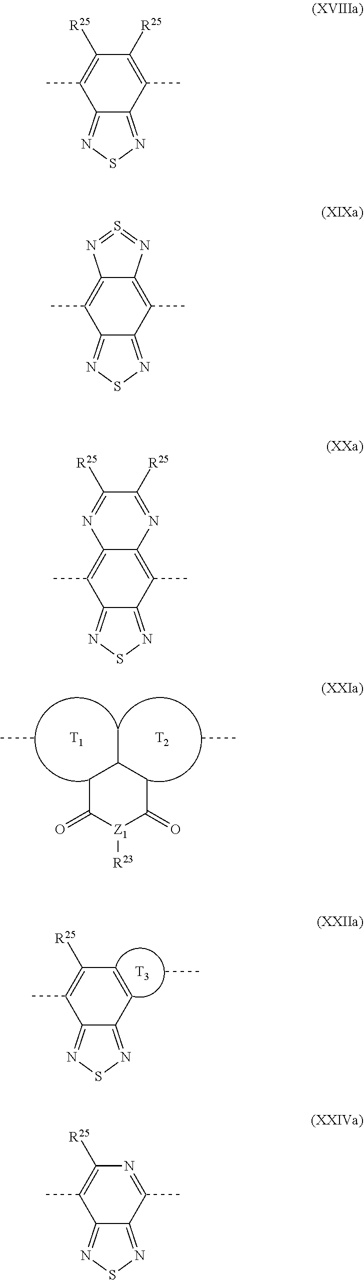

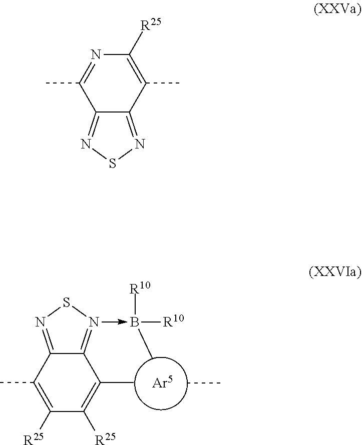

R²³ in each occurrence is a substituent, optionally C₁₋₁₂ alkyl wherein one or more non-adjacent, non-terminal C atoms may be replaced with O, S, COO or CO and one or more H atoms of the alkyl may be replaced with F.

R²⁵ in each occurrence is independently H; F; C₁₋₁₂ alkyl wherein one or more non-adjacent, non-terminal C atoms may be replaced with O, S, COO or CO and one or more H atoms of the alkyl may be replaced with F; or an aromatic group Ar², optionally phenyl, which is unsubstituted or substituted with one or more substituents selected from F and C₁₋₁₂ alkyl wherein one or more non-adjacent, non-terminal C atoms may be replaced with O, S, COO or CO.

Z¹ is Nor P.

T¹, T² and T³ each independently represent an aryl or a heteroaryl ring, optionally benzene, which may be fused to one or more further rings. Substituents of T¹, T² and T³, where present, are optionally selected from non-H groups of R²⁵.

R¹⁰ in each occurrence is a substituent, preferably a C₁₋₂₀ hydrocarbyl group.

Ar⁵ is an arylene or heteroarylene group, optionally thiophene, fluorene or phenylene, which may be unsubstituted or substituted with one or more substituents, optionally one or more non-H groups selected from R²⁵.

Exemplary donor materials are disclosed in, for example, WO2013/051676, the contents of which are incorporated herein by reference.

Electron Acceptor Material

In the case where the material comprising the group of formula (I) is an electron-donor material, it may be used with any electron accepting material containing a group of formula (I) or any other electron accepting material known to the person skilled in the art.

Exemplary electron-accepting materials are non-fullerene acceptors, which may or may not contain a unit of formula (I), and fullerenes.

Exemplary electron-accepting compounds containing at least one unit of formula (I) include:

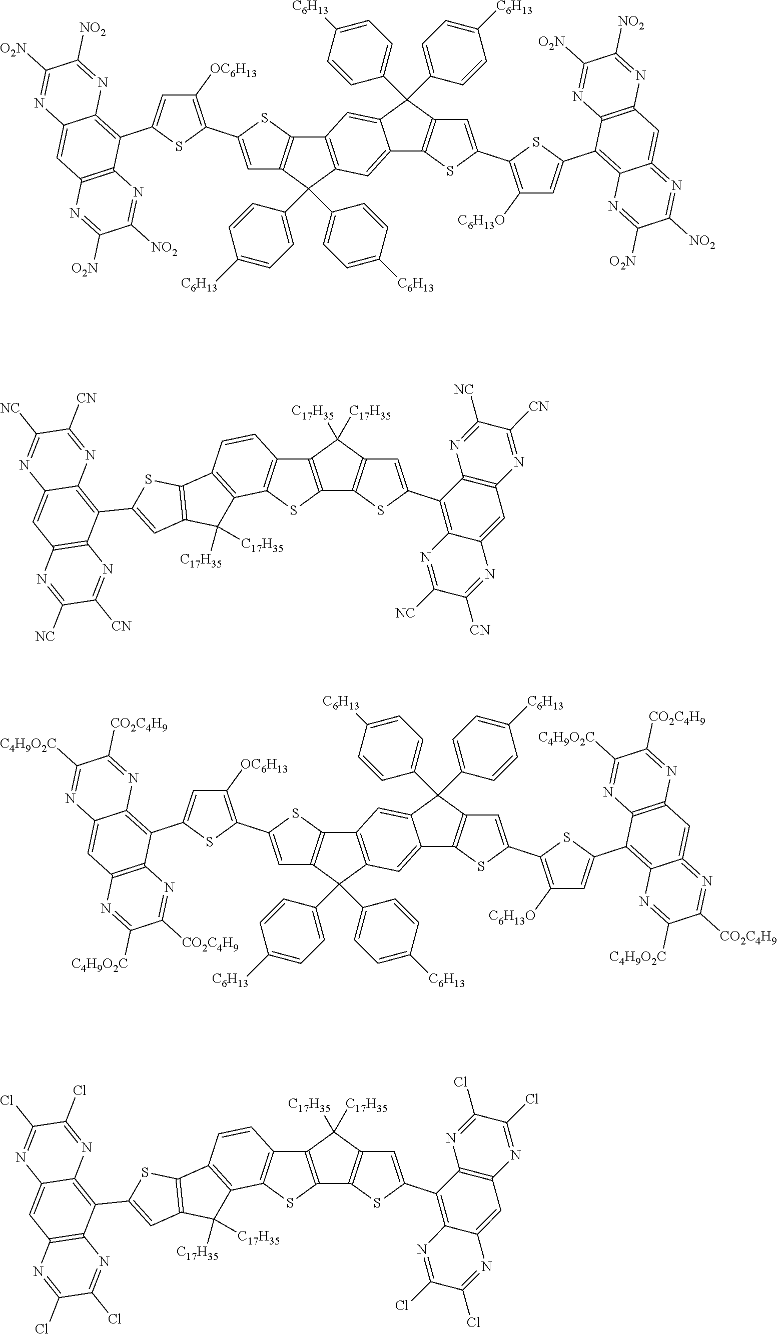

Non-fullerene acceptors which do not contain a unit of formula (I) are described in, for example, Cheng et al, “Next-generation organic photovoltaics based on non-fullerene acceptors”, Nature Photonics volume 12, pages 131-142 (2018), the contents of which are incorporated herein by reference, and which include, without limitation, PDI, ITIC, IEICO and derivatives thereof.

Exemplary fullerene electron acceptor materials are C₆₀, C₇₀, C₇₆, C₇₈ and C₈₄ fullerenes or a derivative thereof including, without limitation, PCBM-type fullerene derivatives (including phenyl-C61-butyric acid methyl ester (C₆₀PCBM), TCBM-type fullerene derivatives (e.g. tolyl-C61-butyric acid methyl ester (C₆₀TCBM)), and ThCBM-type fullerene derivatives (e.g. thienyl-C61-butyric acid methyl ester (C₆₀ThCBM).

Fullerene derivatives may have formula (III):

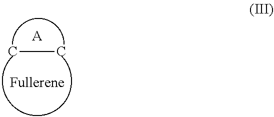

wherein A, together with the C—C group of the fullerene, forms a monocyclic or fused ring group which may be unsubstituted or substituted with one or more substituents.

Exemplary fullerene derivatives include formulae (IIIa), (IIIb) and (IIIc):

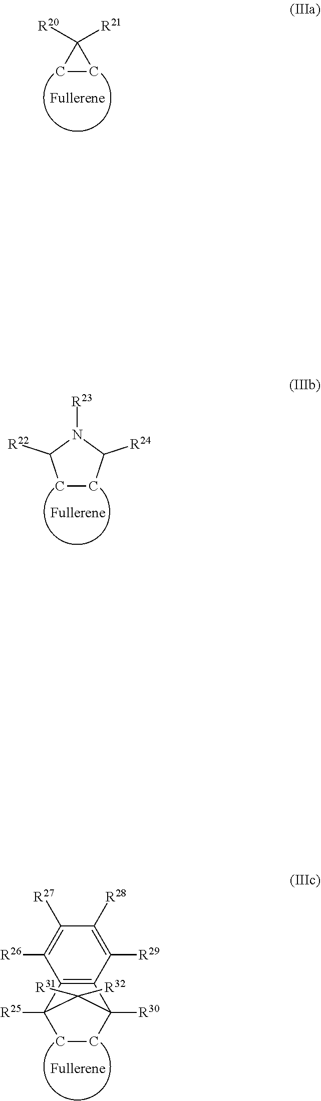

wherein R²⁰-R³² are each independently H or a substituent.

Substituents R²⁰-R³² are optionally and independently in each occurrence selected from the group consisting of aryl or heteroaryl, optionally phenyl, which may be unsubstituted or substituted with one or more substituents; and C₁₋₂₀ alkyl wherein one or more non-adjacent, non-terminal C atoms may be replaced with O, S, CO or COO and one or more H atoms may be replaced with F.

Substituents of aryl or heteroaryl, where present, are optionally selected from C₁₋₁₂ alkyl wherein one or more non-adjacent, non-terminal C atoms may be replaced with O, S, CO or COO and one or more H atoms may be replaced with F.

At least one of the anode and cathode is transparent so that light incident on the device may reach the bulk heterojunction layer. In some embodiments, both of the anode and cathode are transparent.

Electrodes

Each transparent electrode preferably has a transmittance of at least 70%, optionally at least 80%, to wavelengths in the range of 750-1000 nm or 1300-1400 nm. The transmittance may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

Bulk Heterojunction Layer Formation

The bulk heterojunction layer may be formed by any process including, without limitation, thermal evaporation and solution deposition methods.

Preferably, the bulk heterojunction layer is formed by depositing a formulation comprising the electron donor material(s), the electron acceptor material(s) and any other components of the bulk heterojunction layer dissolved or dispersed in a solvent or a mixture of two or more solvents. The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene substituted with one or more substituents selected from chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulation may comprise further components in addition to the electron acceptor, the electron donor and the one or more solvents. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

Applications

A circuit may comprise the OPD connected to a voltage source for applying a reverse bias to the device and/or a device configured to measure photocurrent. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 900 nm, optionally in the range of 900-1000 nm. In some embodiments, the light source has a peak wavelength greater than 1000 nm, optionally in the range of 1300-1400 nm. Unless stated otherwise, absorption spectra as described herein are as measured in solution, optionally toluene solution, using a Cary 5000 UV-vis-IR spectrometer.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g. due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor.

### Examples

Synthesis

Intermediates 1-3 and bromination of Intermediate 3 is disclosed in KR20180042966, the contents of which are incorporated herein by reference.

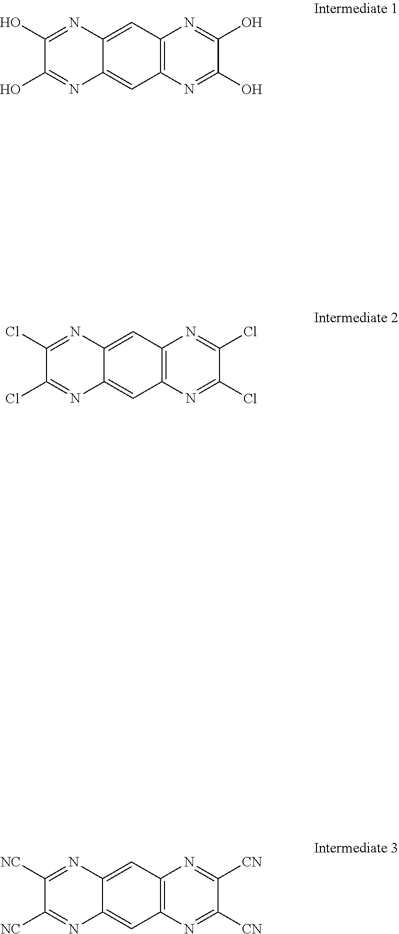 [SMILES: Oc1nc2cc3nc(O)c(O)nc3cc2nc1O ; Clc1nc2cc3nc(Cl)c(Cl)nc3cc2nc1Cl ; N#Cc1nc2cc3nc(C#N)c(C#N)nc3cc2nc1C#N]

Intermediate 4 was prepared according to the following reaction scheme, adapted from Org. Lett. 2011, 13, 6090:

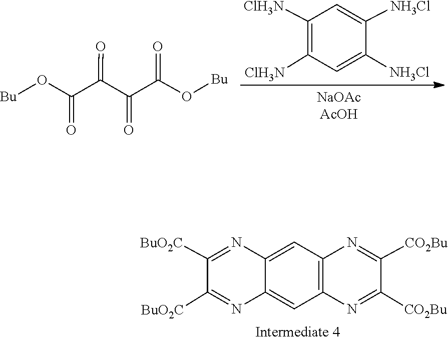

To a mixture of dibutyl-2,3-dioxosuccinate (5.2 g, 20.3 mmol), 1,2,4,5,-benzenetetramine tetrahydrochloride (2.5 g, 8.8 mmol) and sodium acetate (2.9 g, 35.2 mmol) was added glacial acetic acid (135 ml). The mixture was heated to 120° C. under nitrogen in the dark for 16 hours. Volatiles were removed under vacuum, the residue was suspended in chloroform and filtered. To the dark solid was added a second portion of sodium acetate (2.9 g, 35.2 mmol) and glacial acetic acid (135 ml), before again heating to 120° C. under nitrogen in the dark for a further 5 hours. Volatiles were removed under vacuum and the product purified by silica gel column chromatography eluting with ethanol-stabilised chloroform to obtain the product as a yellow solid (1.4 g, 28%).

¹H NMR (600 MHz, CDCl₃, 298 K): δ 1.00 (t, ³J=7.4 Hz, 12H); 1.51 (m, 8H); 1.85 (m, 8H); 4.52 (t, 3J=6.8 Hz, 8H); 9.22 (s, 2H) ppm. LC-MS (ESI, +ve, MeCN/H₂O) m/z: 583.1329 (100%) [MH]⁺.

The bromination of Intermediate 4 may be achieved using the published conditions in Schulz et al., Macromolecules 2013, 46, 6, 2141-2151 without further modification.

The brominated intermediates may be polymerised or coupled to donor groups by methods known to the skilled person, for example Suzuki or Stille coupling or polymerisation.

### Modelling Example 1

All modelling as described in these examples was performed using Gaussian09 software available from Gaussian using Gaussian09 with B3LYP (functional).

HOMO and LUMO levels for range of acceptor (A) of model compounds of General Formula 1 were modelled:

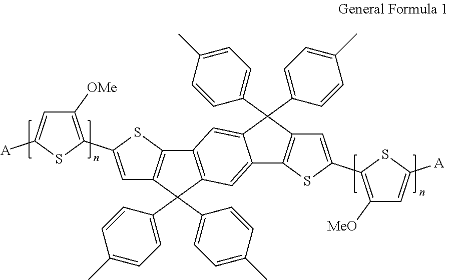

TABLE 1

| Model | | | HOMO | LUMO | Eg | Abs |
|---|---|---|---|---|---|---|
| Compound | Acceptor (A) | n | (eV) | (eV) | (eV) | (nm) |
| 1A (Comparative) | 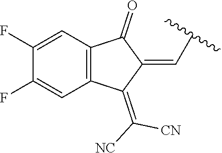 [SMILES: */C=C1\C(=O)c2cc(F)c(F)cc2C1=C(C#N)C#N] | 0 | −5.78 | −3.57 | 2.21 | 562 |
| IB (Comparative) | 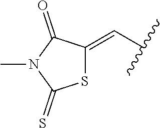 [SMILES: */C=C1\SC(=S)N(C)C1=O] | 1 | −4.81 | −2.83 | 1.98 | 627 |
| 1C (Comparative) | 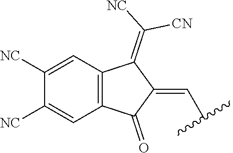 [SMILES: */C=C1\C(=O)c2cc(C#N)c(C#N)cc2C1=C(C#N)C#N] | 1 | −5.44 | −3.81 | 1.62 | 765 |
| ID (Comparative) | 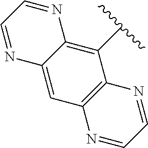 [SMILES: *c1c2nccnc2cc2nccnc12] | 1 | −4.38 | −2.97 | 1.41 | 875 |
| IE (Comparative) | 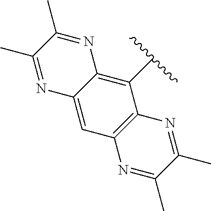 | 1 | −4.20 | −2.59 | 1.61 | 771 |
| IF (Exemplary) | 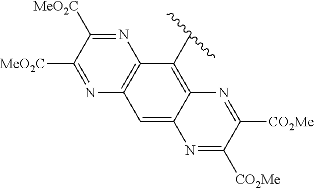 [SMILES: *c1c2nc(C(=O)OC)c(C(=O)OC)nc2cc2nc(C(=O)OC)c(C(=O)OC)nc12] | 1 | −4.67 | −3.39 | 1.28 | 969 |
| IG (Exemplary) | 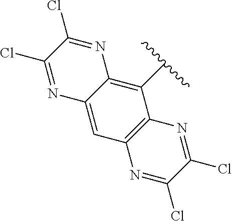 [SMILES: *c1c2nc(Cl)c(Cl)nc2cc2nc(Cl)c(Cl)nc12] | 1 | −4.73 | −3.47 | 1.27 | 979 |
| 1H (Exemplary) | 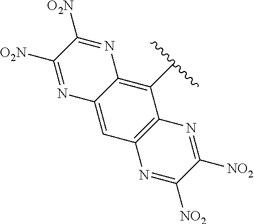 [SMILES: *c1c2nc([N+](=O)[O-])c([N+](=O)[O-])nc2cc2nc([N+](=O)[O-])c([N+](=O)[O-])nc12] | 1 | −5.53 | −4.53 | 1.00 | 1235 |
| 1I (Exemplary) | 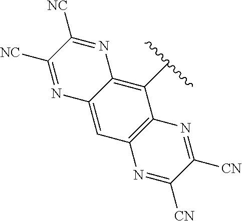 [SMILES: *c1c2nc(C#N)c(C#N)nc2cc2nc(C#N)c(C#N)nc12] | 1 | −5.38 | −4.42 | 0.97 | 1287 |
| 1J (Exemplary) | 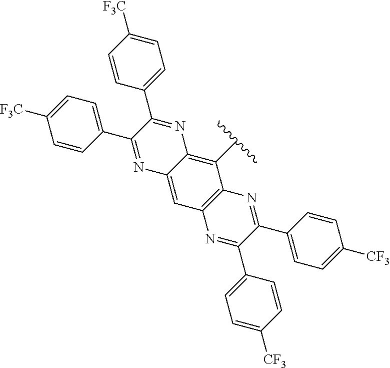 [SMILES: *c1c2nc(-c3ccc(C(F)(F)F)cc3)c(-c3ccc(C(F)(F)F)cc3)nc2cc2nc(-c3ccc(C(F)(F)F)cc3)c(-c3ccc(C(F)(F)F)cc3)nc12] | 1 | −4.59 | −3.31 | 1.28 | 972 |
| 1K (Exemplary) | 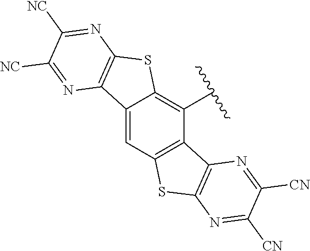 [SMILES: *c1c2sc3nc(C#N)c(C#N)nc3c2cc2sc3nc(C#N)c(C#N)nc3c12] | 1 | −4.90 | −3.83 | 1.07 | 1154 |
| IL (Exemplary) | 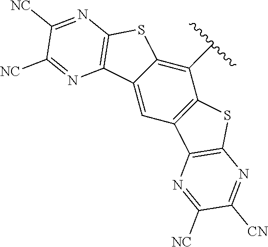 [SMILES: *c1c2sc3nc(C#N)c(C#N)nc3c2cc2c1sc1nc(C#N)c(C#N)nc12] | 1 | −5.11 | −3.51 | 1.60 | 775 |

### Modelling Example 2

The effect of a range of groups X on the HOMO and LUMO of and the effect of the presence of Ar¹ and Ar² on HOMO and LUMO of materials of formula (I) was modelled using model compounds of General Formula 2:

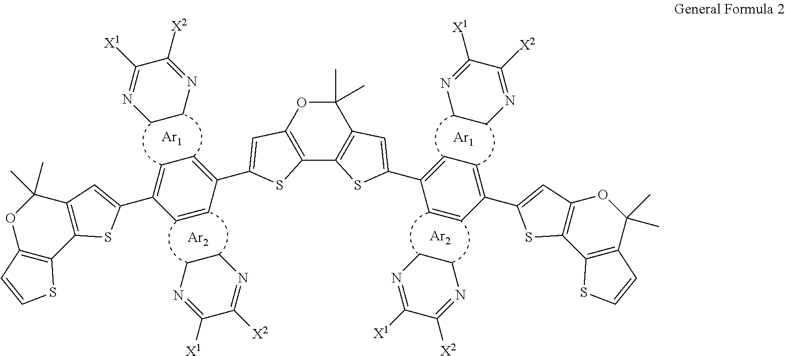

TABLE 2

| X¹ | X² | Ar1/Ar2 | HOMO (eV) | LUMO (eV) | Eg (eV) | Abs (nm) |
|---|---|---|---|---|---|---|
| H | H | — | −4.31 | −3.16 | 1.16 | 1071 |
| Cl | Cl | — | −4.67 | −3.58 | 1.08 | 1146 |
| CN | Me | — | −4.66 | −3.69 | 0.97 | 1274 |
| CO2Me | CO2Me | — | −4.45 | −3.48 | 0.96 | 1286 |
| NO2 | NO2 | — | −5.15 | −4.43 | 0.71 | 1739 |
| CN | CN | — | −5.10 | −4.40 | 0.70 | 1779 |
| CN | CN | 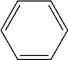 | −5.64 | −4.39 | 1.25 | 992 |
| CF₃ | CF₃ | — | −4.69 | −3.44 | 1.25 | 990 |
| 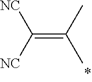 | 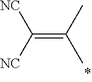 | — | −5.32 | −4.37 | 0.95 | 1306 |
| CN | CN | 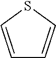 | −5.51 | −3.79 | 1.72 | 720 |

### Modelling Example 3

The effect of the electron-accepting unit of formula (I) on LUMO and band gap of model compounds of General Formula 3, in which Acc is the electron-accepting unit, is shown in Table 3:

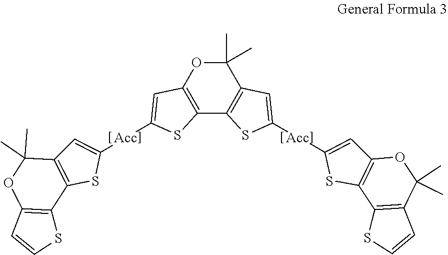

TABLE 3

| Model | | HOMO | LUMO | | Abs |
|---|---|---|---|---|---|
| Compound | Acceptor | (eV) | (eV) | Eg (eV) | (nm) |
| 3A (comparative) | 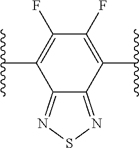 [SMILES: *c1c(F)c(F)c(*)c2nsnc12] | −4.65 | −2.92 | 1.73 | 716 |
| 3B (comparative | 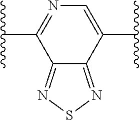 [SMILES: *c1cnc(*)c2nsnc12] | −4.60 | −3.06 | 1.54 | 803 |
| 3C (Exemplary) | 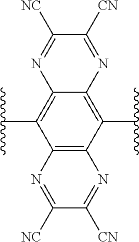 [SMILES: *c1c2nc(C#N)c(C#N)nc2c(*)c2nc(C#N)c(C#N)nc12] | −5.10 | −4.40 | 0.70 | 178 |

1. A material comprising an electron-accepting unit of formula (I): 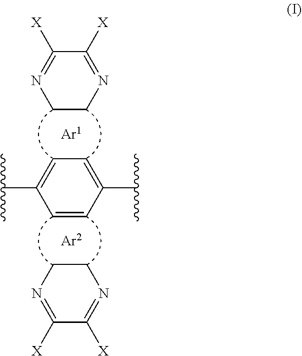
wherein Ar¹ is a 5- or 6-membered aromatic or heteroaromatic ring or is absent; Ar² is a 5- or 6-membered aromatic or heteroaromatic ring or is absent; and each X is independently H or a substituent with the proviso that at least one X is an electron-withdrawing group and wherein X groups bound to adjacent carbon atoms may be linked to form an electron-withdrawing group; the material further comprising an electron-donating unit D comprising a fused or unfused furan or thiophene.

2. The material according to claim 1 wherein each X is an electron-withdrawing group.

3. The material according to claim 1 wherein the or each electron-withdrawing group is independently selected from:
a group R⁴ wherein each R⁴ is independently selected from the Cl, CN, NO₂, COOR³, C₁₋₆ fluoroalkyl, e.g. —CF₃, —OR³, —SR³, —SO₂R³, —SO₃R³, —CHO, —C(O)R³, —C(S)R³, —C(S)OR³, —OC(O)R³, —OC(S)R³, —C(O)SR³, —SC(O)R³, —C(O)NR³₂, —NRC(O)R³, —CH═CH(CN), —CH═C(CN)₂, —C(CN)═C(CN)₂, —CH═C(CN)(R³), —CH═C(CN)C(O)OR³ and —CH═C(CONR³²)₂, wherein R³ is H or a substituent; and
phenyl substituted with one or more R⁴ groups.

4. The material according to claim 1 wherein the material is a non-polymeric compound.

5. The material according to claim 4 wherein the material has formula (Ia) or (Ib): 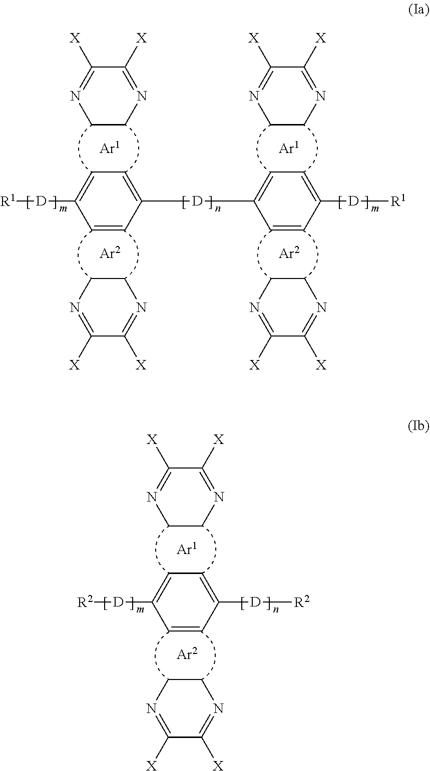
wherein n is at least 1; m is 0, 1, 2 or 3; D in each occurrence is independently an electron-donating unit comprising a fused or unfused thiophene or furan which may be unsubstituted or substituted with one or more substituents; and R¹ and R² independently in each occurrence is H or a substituent.

6. The material according to claim 1 wherein the material is a polymer; the unit of formula (I) is an electron-accepting repeat unit of formula (I); and the electron-donating unit D is an electron-donating repeat unit.

7. The material according to claim 1 wherein D is selected from formulae (IIa)-(IIo): 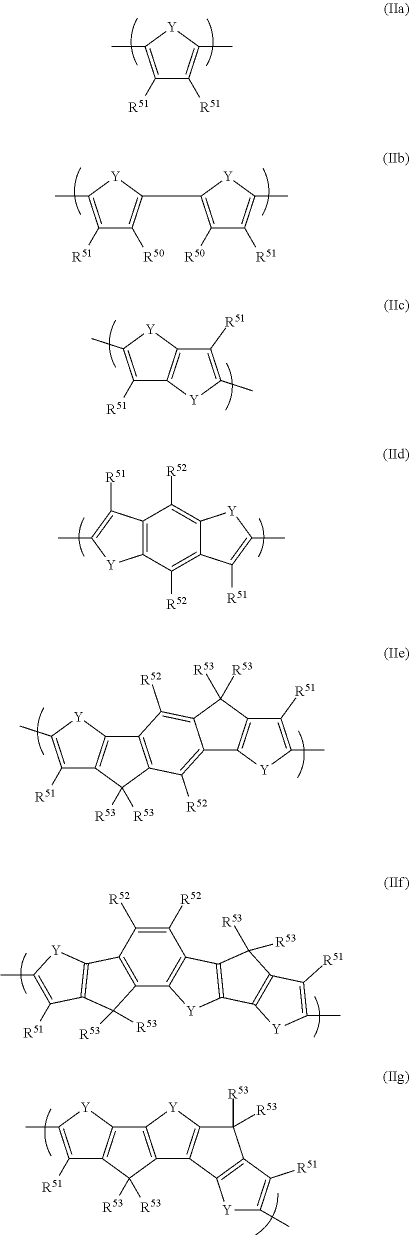 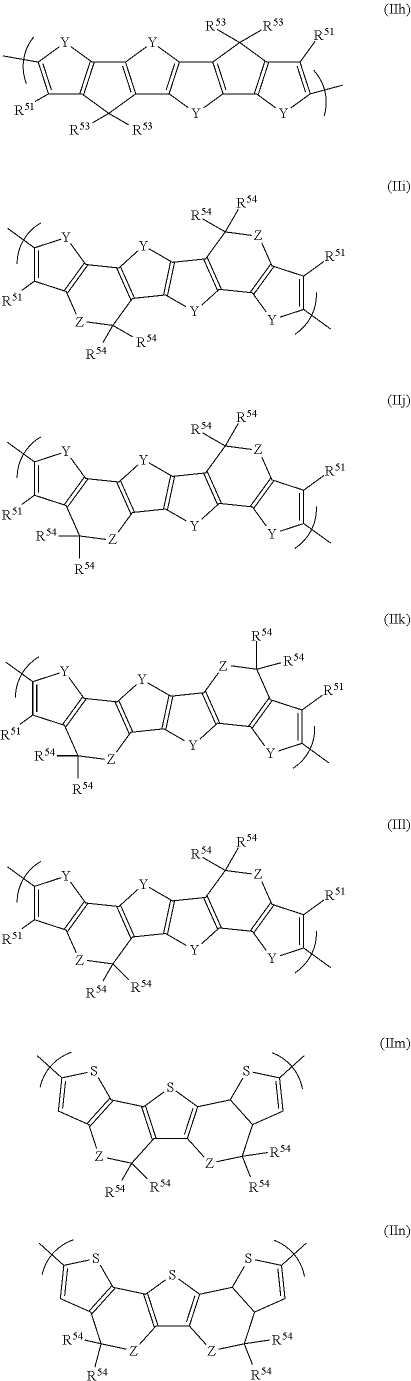 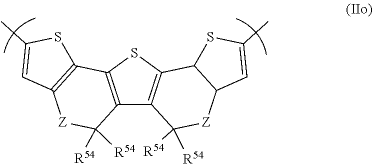
wherein Y in each occurrence is independently O or S, Z in each occurrence is O, S, NR⁵⁵ or C(R⁴)₂; R⁵⁰, R⁵¹, R⁵², R⁵⁴ and R⁵⁵ independently in each occurrence is H or a substituent wherein R⁵⁰ groups may be linked to form a ring; and R⁵³ independently in each occurrence is a substituent.

8. A polymer comprising a repeat unit of formula (I): 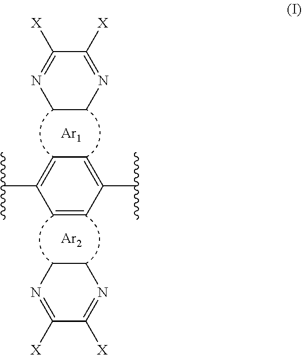
wherein Ar¹ is a 6-membered aromatic or heteroaromatic ring or is absent; Ar² is a 6-membered aromatic or heteroaromatic ring or is absent; and each X is independently H or a substituent with the proviso that at least one X is an electron-withdrawing group and wherein X groups bound to adjacent carbon atoms may be linked to form an electron-withdrawing group.

9. A composition comprising an electron donor and an electron acceptor wherein at least one of the electron donor and electron acceptor is a material according to claim 1.

10. The composition according to claim 9 wherein the electron acceptor is the material comprising an electron-accepting unit of formula (I).

11. The composition according to claim 10 wherein the electron acceptor is a non-polymeric compound.

12. The composition according to claim 9 wherein the electron donor is the material comprising an electron-accepting unit of formula (I) or the polymer comprising a repeat unit of formula (I).

13. The composition according to claim 12 wherein the electron donor is the polymer when the material is a polymer, the unit of formula (I) is an electron-accepting repeat unit of formula (I), and the electron-donating unit D is an electron-donating repeat unit or the polymer comprising a repeat unit of formula (I).

14. An organic electronic device comprising an active layer comprising the material according to claim 1.

15. An organic electronic device according to claim 14 wherein the organic electronic device is an organic photoresponsive device comprising a bulk heterojunction layer disposed between an anode and a cathode and wherein the bulk heterojunction layer comprises a composition comprising an electron donor and an electron acceptor wherein at least one of the electron donor and electron acceptor is a material comprising an electron-accepting unit of formula (I).

16. An organic electronic device according to claim 15 wherein the organic photoresponsive device is an organic photodetector.

17. A photosensor comprising a light source and an organic photodetector according to claim 16, wherein the photosensor is configured to detect light emitted from a light source.

18. A photosensor according to claim 17, wherein the light source emits light having a peak wavelength of at least 900 nm.

19. A formulation comprising a material, according to claim 1 dissolved or dispersed in one or more solvents.

20. A method of forming an organic electronic device according to claim 14 wherein formation of the active layer comprises deposition of a formulation comprising the material dissolved or dispersed in one or more solvents onto a surface and evaporation of the one or more solvents.
